(19) United States
(12) Patent Application Publication
(10) Pub. No.: US 2014/0206007 A1
(43) Pub. Date: Jul. 24, 2014

(54) ELECTROMAGNETICALLY ACTUATED DROPLET MICROFLUIDIC CHIP AND SYSTEM

(71) Applicant: THE JOHNS HOPKINS UNIVERSITY, Baltimore, MD (US)

(72) Inventors: Tza-Huei WANG, Timonium, MD (US); Chi-Han Chiou, Baltimore, MD (US); Dong Jin Shin, Baltimore, MD (US)

(73) Assignee: The Johns Hopkins University, Baltimore, MD (US)

(21) Appl. No.: 13/745,511

(22) Filed: Jan. 18, 2013

(51) Int. Cl. C12Q 1/68

(52) U.S. Cl. CPC C12Q 1/6806
USPC 435/6.12; 435/287.2

(57) ABSTRACT

A microfluidic system includes a microfluidic cartridge and an electromagnetic droplet actuator arranged proximate the microfluidic cartridge. The microfluidic cartridge includes a plurality of droplet wells with topological barrier structures between adjacent wells. The topological barrier structures are configured to allow magnetic particles and material attached to the magnetic particles to pass between adjacent wells while confining droplets within respective wells. The electromagnetic droplet actuator includes a plurality of electromagnetic components arranged to provide an electronically selectable magnetic field pattern to actuate movement of a plurality of magnetic particles when contained within at least one droplet in at least one of the plurality of droplet wells.

### FEDERAL FUNDING BY THE U.S. GOVERNMENT

This invention was made with Government support of Grant Nos. U54CA151838 and R01CA155305, awarded by the Department of Health and Human Services, The National Institutes of Health (NIH). The U.S. Government has certain rights in this invention.

### BACKGROUND

1. Field of Invention

The field of the currently claimed embodiments of this invention relates to microfluidic systems, and more particularly to electromagnetically actuated microfluidic systems.

2. Discussion of Related Art

Nucleic acids-based diagnostics is a fast-growing field encompassing many applications including infectious diseases, oncology, pharmacogenomics and genetic testing. Microfluidic technologies used to create miniaturized total analysis systems (μTAS) have demonstrated the potential to move nucleic acids-based diagnostic tools to the front lines of health care in the past two decades.¹ Several examples of continuous flow microfluidic technologies have already demonstrated the ability to carry out nucleic acid extraction, amplification and detection for genetic assays on a single platform.²⁻⁷ Nevertheless, challenges remain before these platforms can be adopted in clinical settings. One often-cited problem is the difficulty of interfacing real-world biological samples with microfluidic platforms.^{8,9} Another major challenge is the reliance on sophisticated instruments that are not amenable to scaling down for the use outside the laboratory setting, which inhibits many technologies from developing beyond the status of ‘chip-in-a-lab’.^{8,9} In light of these challenges, droplet-based platforms using droplets for storage and processing of reagents on the order of microliters have demonstrated the potential to bring μTAS closer to clinics. Sample routing in these platforms is typically pumpless and valveless, providing ease in integration and greater flexibility for handling various physiological samples and assays.^{10,11}

Droplet-based microfluidic devices using dielectrophoretic,¹²⁻¹⁵ electrowetting,^{11,13,16-22} and magnetic²³⁻³³ actuation mechanisms have made significant progress towards integrating essential manipulation for bioanalysis such as transport, mixing, splitting, and merging of discrete droplets. In particular, magnetic particle-based platforms are readily compatible with biomolecule extraction from crude samples using various commercially available reagents and surface functionalized particles, whereas purely dielectrophoretic or electrowetting modes of operation are less readily suited.¹¹ Magnetic particle-based droplet manipulation has been combined with silica-coated magnetic bead enabled solid phase extraction protocols to perform DNA extraction and purification on chip.^{26,29,30,32} Movable permanent magnets have been used to successfully perform transport, splitting, and merging of droplets for genetic assays.^{26,28,29} However, such platforms require the use of costly precision translational stages in order to automate particle actuation. Moreover, permanent magnet-actuated platforms generally lack strategies for bead agitation, which may substantially decrease the binding/washing efficiency.²⁹ Alternatively, planar coils have been demonstrated to be capable of generating effective magnetic fields for long-range transport and manipulations of droplets,^{23,31} providing a simplified apparatus for droplet operations.

Among all droplet manipulation steps, splitting of magnetic particles from the parent droplet presents the greatest challenge because it dependents on multiple factors, including capillary force, magnetic particle load, and magnet velocity.³⁴ Droplet immobilization provides a simpler approach to this challenge. Successful splitting is often contingent on effective immobilization of parent droplet, and multiple strategies have been discussed. Typical strategies include substrate patterning^{26,32,33} or placing physical barriers.^{27,29} The first strategy involves local patterning of hydrophilic spots in order to anchor the aqueous droplets on a flat surface. However, such patterns also have affinity towards biomolecules and particles, resulting in adsorption of sample molecules as well as increased friction on magnetic particles. Following the alternative strategy, we recently reported a surface topography assisted droplet splitting based on a permanent magnet platform, where the elevated structures on chip improved the robustness of dissociating magnetic particles from the parent droplets.²⁹ However, each of the above-noted approaches have limitations with respect to commercial usefulness. There thus remains a need for improved microfluidic chips and systems.

### SUMMARY

A microfluidic system according to an embodiment of the current invention includes a microfluidic cartridge and an electromagnetic droplet actuator arranged proximate the microfluidic cartridge. The microfluidic cartridge includes a plurality of droplet wells with topological barrier structures between adjacent wells. The topological barrier structures are configured to allow magnetic particles and material attached to the magnetic particles to pass between adjacent wells while confining droplets within respective wells. The electromagnetic droplet actuator includes a plurality of electromagnetic components arranged to provide an electronically selectable magnetic field pattern to actuate movement of a plurality of magnetic particles when contained within at least one droplet in at least one of the plurality of droplet wells.

A method of processing a sample according to an embodiment of the current invention includes providing a droplet containing the sample and a plurality of magnetic particles in a droplet well of a microfluidic cartridge. The microfluidic cartridge includes a plurality of droplet wells with topological barrier structures between adjacent wells. The method further includes applying a magnetic field pattern to actuate movement of the plurality of magnetic particles when contained within at least one droplet in at least one of the plurality of droplet wells. The topological barrier structures are configured to allow magnetic particles and material attached to the magnetic particles to pass between adjacent wells while confining droplets within respective wells.

### BRIEF DESCRIPTION OF THE DRAWINGS

Further objectives and advantages will become apparent from a consideration of the description, drawings, and examples.

FIG. 1A is a photograph of a disposable chip containing aqueous food dyes pre-injected in each compartment according to an embodiment of the current invention. FIG. 1B is a schematic illustration of a magnetic droplet manipulation system according to an embodiment of the current invention that includes a disposable chip and actuation module for automated nucleic acid extraction. FIG. 1C is a diagram illustrating alignment of droplet cartridge on coil PCB according to an embodiment of the current invention. Top layer of coils were centered at each droplet compartments, which would automatically align the bottom layer of coils (black, dashed lines) to the centers of sieve structures. FIG. 1D shows layout of droplet cartridge and sample processing order indicated by black arrows. Droplet cartridge has seven compartments separated by sieve structures to facilitate droplet splitting. Each compartment was primed with reagent droplets and overlaid in mineral oil. The magnetic particles were split from the lysis/binding buffer droplet and transferred to washing buffers WB1a, WB1b, WB2a, and WB2b for DNA extraction. Afterwards, the particle-bound DNA was directly eluted in PCR reagent droplet, followed by extraction of particles into the last compartment.

FIG. 2A is a schematic illustration of topography-assisted droplet splitting. Magnetic particle cluster was extruded from the parent droplet as the magnetic attraction moved the cluster through a sieve structure. A wide-range field gradient was generated by creating magnetic fields of opposing polarity on two adjacent coils. FIG. 2B shows simulation of the coil-generated magnetic field topology with one positive phase and one negative phase. The simulated magnetic field gradient between two adjacent coils on the same layer was around 4 T/m at an elevation of 100 μm and coil input current of 3 A.

FIGS. 3A and 3B show magnetic field gradient simulations illustrating two modes of operation for magnetic particle manipulation according to an embodiment of the current invention. (A) Splitting is achieved by applying a strong field gradient between two droplet compartments. Negative, zero and positive polarity currents are applied in 3 consecutive coils to generate field maximum at the center of destination droplet and field minimum at the center of current droplet. The resulting force applied on magnetic particles facilitates droplet splitting. (B) Agitation is achieved by alternating between two polarities of current applied to a single coil directly below the droplet containing magnetic particles. When the field is directed upward, gradient maximum is located at the center of the droplet and particles are concentrated. In reverse polarity, gradient is directed away from the center of the droplet and force is applied on particles to move towards the fringes of the droplet.

FIGS. 4A-4F is a photographic sequence showing droplet splitting and merging assisted by a sieve structure according to an embodiment of the current invention. From left to right: (A) magnetic particles were dispensed into the parent droplet containing yellow food dye, forming columns due to magnetization under large transverse B-field; (B) magnetic particles collected towards the maximum of coil-generated field gradient, located at the center of the sieve structure; (C) field gradient maximum was changed to the center of the adjacent droplet, causing magnetic particles to pull through the sieve structure; (D) this process formed a long neck, which reduces capillary force until a sessile plug containing magnetic particles was formed; (E-F) the separated plug was magnetically transported and merged with the adjacent droplet.

FIGS. 5A-5C (top, middle and lower rows, respectively) are photographic sequences showing droplet agitation. (A) Left: magnetic particles attracted towards the center of the droplet. Center: particles dispersed towards the fringes of the droplet. Right: particles dispersed inside the droplet when no current is applied to the coil. (B) Mixing of blue food dye in water droplet with the assistance of agitation. (C) Diffusion of blue food dye in water droplet in absence of agitation.

FIGS. 6A and 6B show an example of on-chip droplet manipulation for sample processing according to an embodiment of the current invention. All images were converted to gray scale for analysis. FIG. 6A Photographs of sequential splitting, merging, and washing performed in an automated fashion. The arrows indicate magnetic particle clusters. FIG. 6B Potential carryover of PCR inhibitors can be minimized by successive washing steps. Droplet manipulation in the demonstration above was performed with 300 μg particle load and 15 μL reagent droplets each.

FIGS. 7A and 7B show amplification signals obtained from PCR on chip. FIG. 7A Real time PCR amplification curve. Amplification curve (black solid line) is generated by smoothing raw signal (0) with 8th order Savitsky-Golay filter. FIG. 7B Gel electrophoresis image of PCR products. The amplified products (Lane 1) were verified by comparing against positive controls (Lane 2) and no-template controls (Lane 3) amplified from male genomic DNA on a conventional thermal cycler using 2% agarose gel electrophoresis. Brighter product band in Lane 1 compared to Lane 2 indicates higher product yield due to the lower denaturation temperature for on-chip thermal cycling. Products are 167 bp in length.

FIGS. 8A and 8B are photograph of devices fabricated using (A) PDMS cast from ABS mold and (B) PMMA machined using a laser cutter.

FIG. 9 shows genomic DNA extraction yield characterization. Human adenocarcinoma cell line culture (Panc 1) was suspended in phosphate buffered saline, quantified using a hemocytometer slide and serially diluted. DNA was extracted in 10 uL elution volume using the electromagnetic bead manipulation platform and quantified using PicoGreen assay (Quant-iT, Invitrogen).

FIG. 10 is a schematic illustration of instrumentation for thermal cycling and optical detection.

FIG. 11A shows characterization of magnetic field generated by a single planar coil on a printed circuit board as a function of driving current. FIG. 11B shows characterization of Joule heating as a function of time, for driving current of 3 A.

FIG. 12 shows an example of a condition where droplet splitting fails. In this case, excessive bead loading results in the entire parent droplet transporting without dissociation of the particles. This problem is addressed by reducing the particle load such that the magnetic particles dissociate before merging with the droplet in next compartment.

FIGS. 13A and 13B show reference data used to correlate buffer color to fraction of lysis/binding buffer (LSB). (A) Standard droplets with known volume fraction of LSB in transparent solution. (B) Plot of standard curve used to correlate normalized mean gray value of droplet to expected fraction of LSB present in the mixture.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

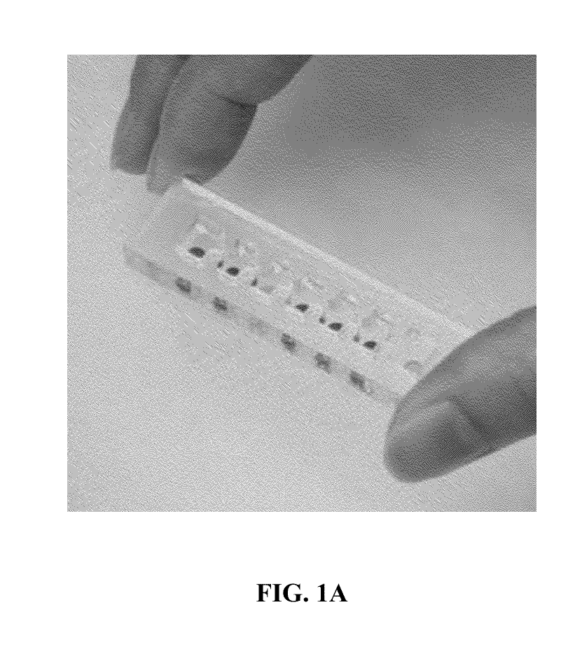

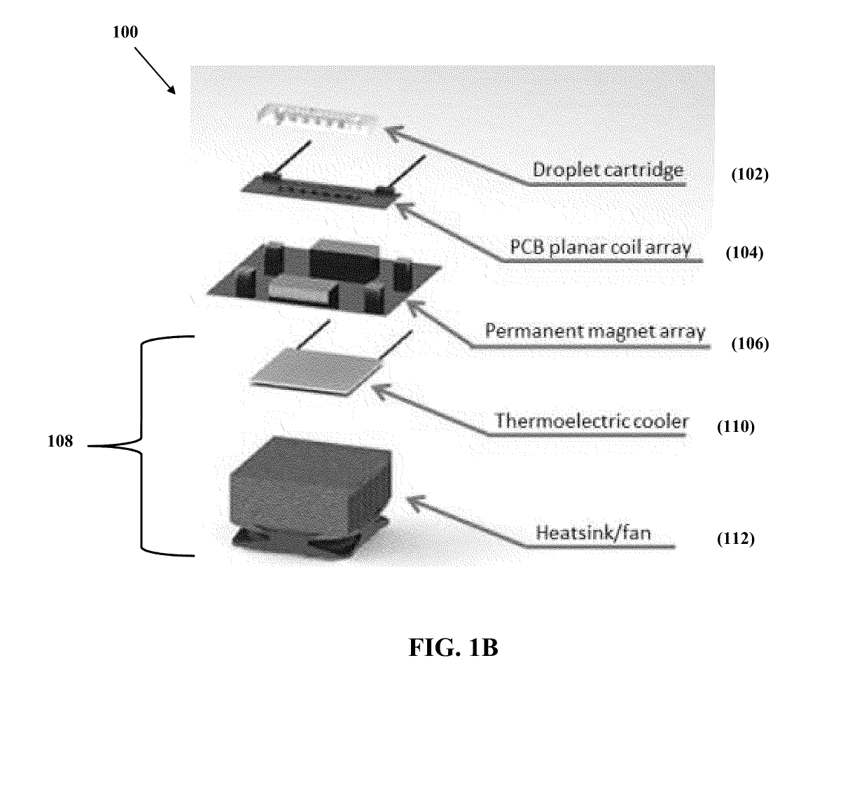

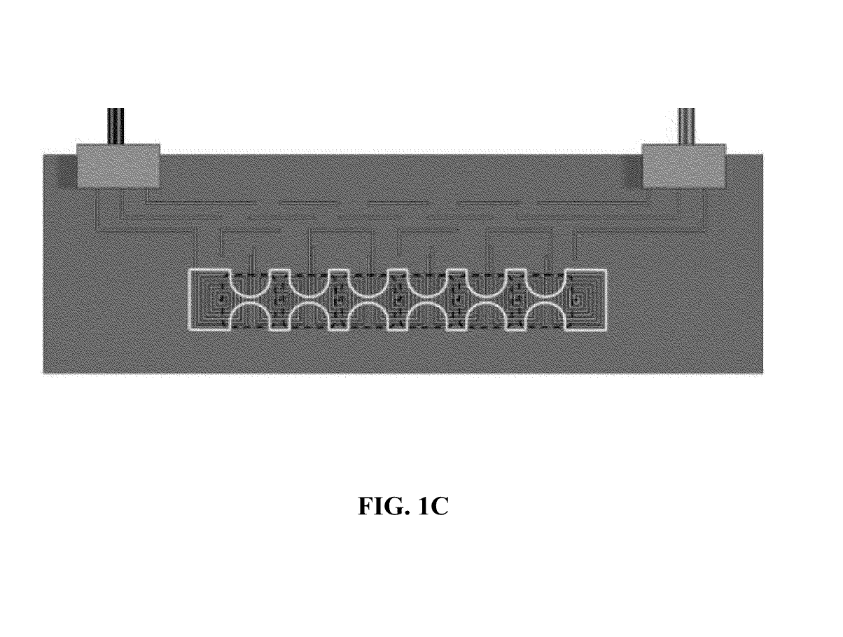

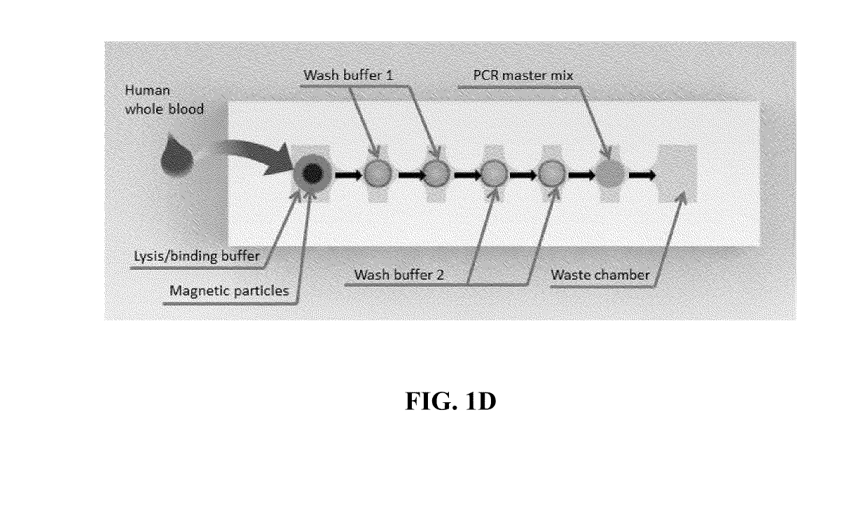

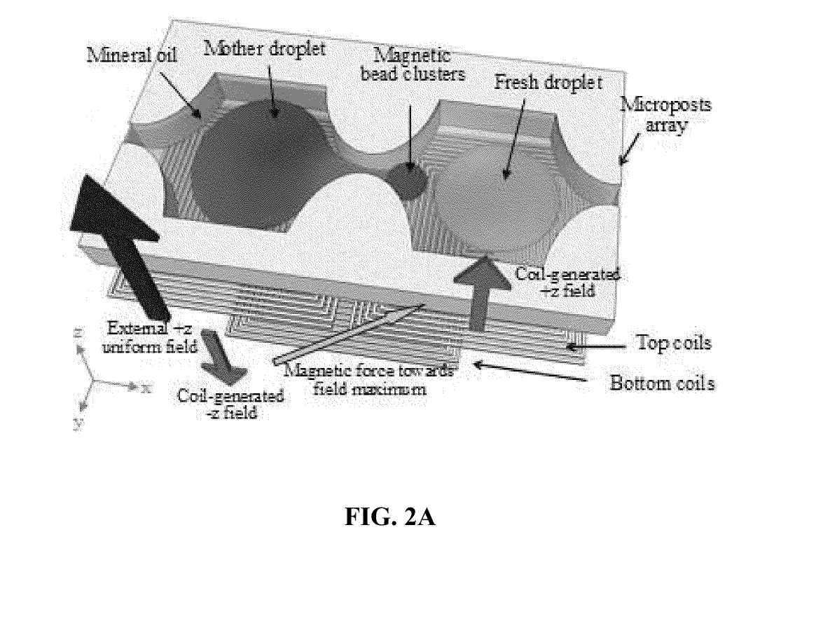

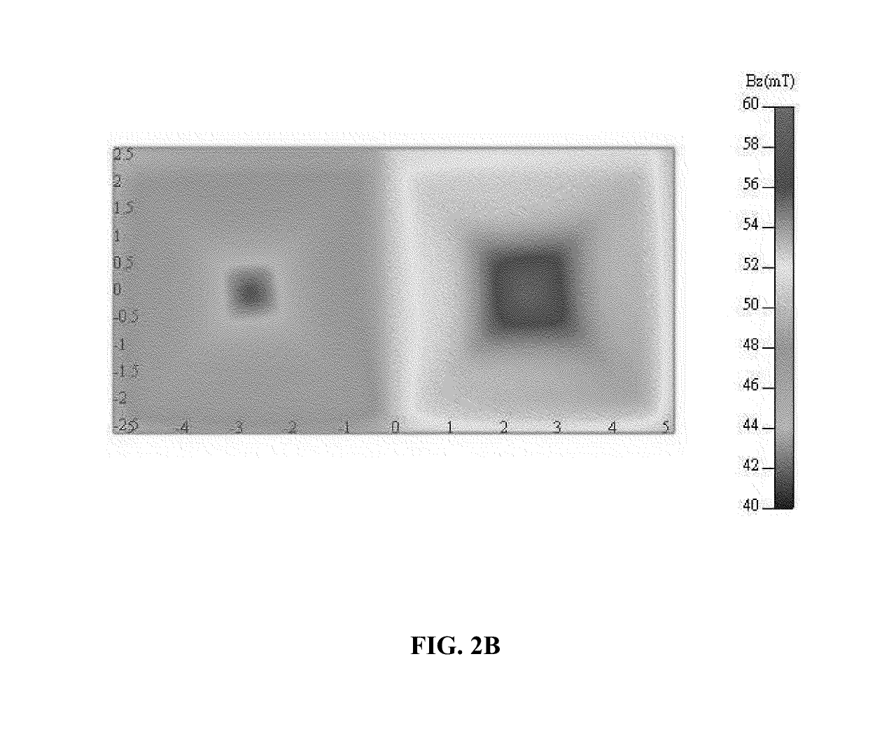

FIGS. 1A-1D show an example of a microfluidic system. FIG. 1B is a schematic illustration of a microfluidic system 100 according to an embodiment of the current invention. The microfluidic system 100 has a microfluidic cartridge 102 and an electromagnetic droplet actuator 104 arranged proximate the microfluidic cartridge 102. The microfluidic cartridge 102, as can be seen more clearly in FIGS. 1A and 1D, includes a plurality of droplet wells with topological barrier structures between adjacent wells. (See also FIG. 2A.) The topological barrier structures are configured to allow magnetic particles and material attached to said magnetic particles to pass between adjacent wells while confining droplets within respective wells. The electromagnetic droplet actuator includes a plurality of electromagnetic components arranged to provide an electronically selectable magnetic field pattern to actuate movement of a plurality of magnetic particles when contained within at least one droplet in at least one of said plurality of droplet wells. Although not shown in FIG. 1B, the electromagnetic droplet actuator 104 can include a power source, a signal generator and other electronics according to the particular application. The electronics can automate, semi-automate and/or otherwise assist the user in actuating movement of the plurality of magnetic particles.

In some embodiments, the electromagnetic droplet actuator 104 is configured to provide a mixing mode such that the plurality of magnetic particles are caused to move with a time-varying pattern within the droplet to cause mixing within the droplet.

In some embodiments, the electromagnetic droplet actuator 104 is configured to provide a separation mode such that the plurality of magnetic particles are caused to move from one droplet well to an adjacent droplet well along with material attached to at least some of the plurality of magnetic particles while each droplet remains confined within a respective droplet well. This can be, for example, droplet splitting. In some embodiments, the electromagnetic droplet actuator 104 can be configured to provide both a separation mode and a mixing mode.

In some embodiments, the plurality of electromagnetic components can be electromagnetic coils arranged such that each droplet well has a corresponding closest electromagnetic coil substantially centered thereon (FIGS. 1C and 2A). In some embodiments, as is illustrated in FIG. 2B, the plurality of electromagnetic components can be configured in two layers, for example. This can allow additional spatial resolution for particle splitting. Because each coil is roughly the size of a compartment chamber, a single-layer design does not generate a field maximum at both the center of the compartment and the junction between compartments. For particle splitting, according to an embodiment of the current invention, the field is first focused at the junction between two compartments using coils from layer 1. Afterwards, coils from layer 2 generate a field maximum at the target compartment and a field minimum at the originating compartment, causing the magnetic particles to split. The electromagnetic coils can be formed on a printed circuit board (PCB), for example. However, the broad concepts of the current invention are not limited to this example.

In some embodiments, the microfluidic system 100 can further include a magnet component 106 arranged proximate the electromagnetic droplet actuator 104 in which the magnet component 106 includes at least one permanent magnet configured to provide a stationary magnetic field component to supplement the electronically selectable magnetic field pattern when produced by the electromagnetic droplet actuator 104.

In some embodiments, the microfluidic system 100 can further include a heat control unit 108 arranged to be in thermal exchange with the microfluidic cartridge 102. The heat control unit 108 can include a thermoelectric cooler 110 in some embodiments. The heat control unit 108 can alternatively, or additionally, include a heat sink 112 and/or a fan.

Another embodiment of the current invention is directed to a method of processing a sample that includes providing a droplet containing the sample and a plurality of magnetic particles in a droplet well of a microfluidic cartridge. The microfluidic cartridge includes a plurality of droplet wells with topological barrier structures between adjacent wells. The method also includes applying a magnetic field pattern to actuate movement of the plurality of magnetic particles when contained within at least one droplet in at least one of the plurality of droplet wells. The topological barrier structures are configured to allow magnetic particles and material attached to the magnetic particles to pass between adjacent wells while confining droplets within respective wells.

In some embodiments, the applying the magnetic field pattern to actuate movement of the plurality of magnetic particles causes the plurality of magnetic particles to move with a time-varying pattern within the droplet to cause mixing within the droplet.

In some embodiments, the applying the magnetic field pattern to actuate movement of the plurality of magnetic particles causes the plurality of magnetic particles to move from one droplet well to an adjacent droplet well along with material attached to at least some of the plurality of magnetic particles.

The following examples describe some embodiments and some applications in more detail. However, the broad concepts of the current invention are not limited to the particular examples.

### Examples

In the following examples an embodiment of the current invention as illustrated in FIGS. 1A-1D is used for PCR-based genetic testing in which magnetic particles are actuated using coil array-induced magnetic field gradients and are assisted by topographical barriers for splitting operations. The combination of electro-magnetic coils and topographical barriers enables simplified, effective and fully automated manipulations of droplets from a wide variety of buffers and reagents—including lysis, binding, wash, elution buffer and PCR reagent—needed for carrying out the entire process of genetic detection assays. We demonstrate the functions of our droplet manipulation platform according to embodiment of the current invention by performing all processing steps between whole blood sample input and real-time amplification detection on a single cartridge. This system demonstrates automated molecular diagnostics on a low-cost disposable cartridge.

### Experimental

Fabrication of the Droplet Cartridge

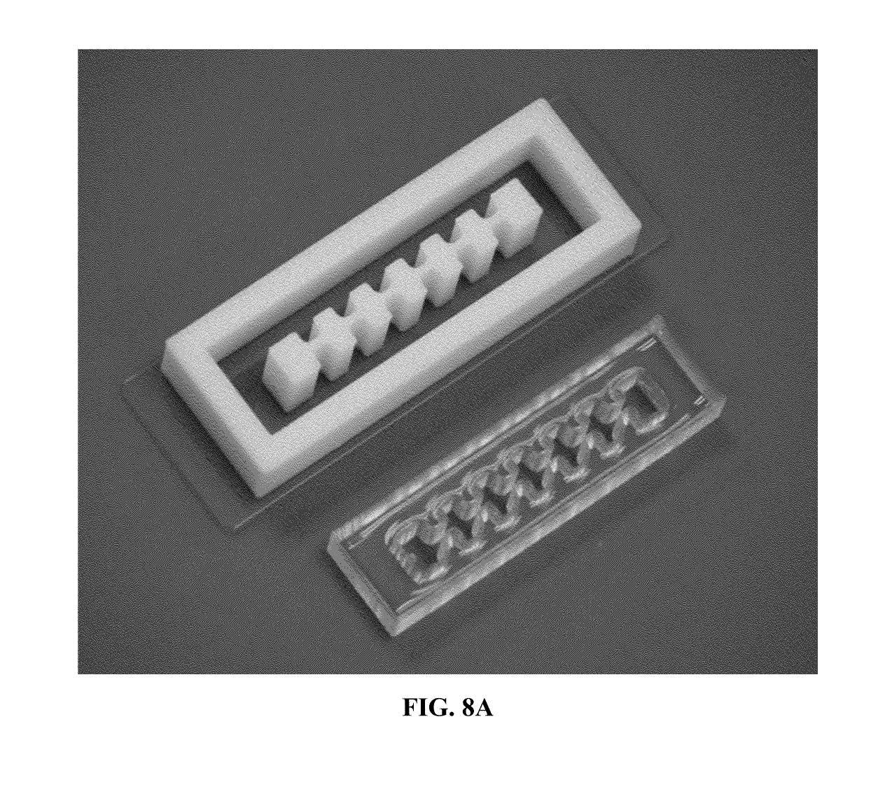

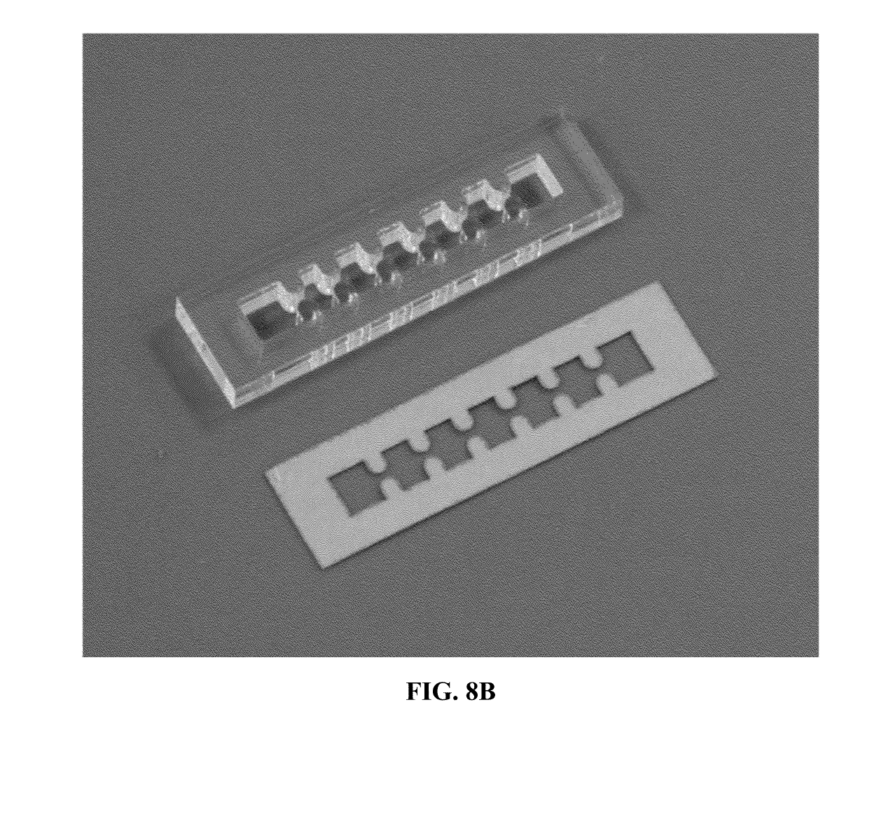

The cartridge consisted of seven compartments connected serially by six sieve structures (see FIGS. 1A and 1D). Each sieve was designed with a radius of curvature of 1.5 mm and a gap width of 750 μm. Outer dimensions of the cartridge were approximately 56×15×6 mm. In this example, we developed two processes for producing these devices. The first method utilized acrylonitrile butadiene styrene (ABS) molds generated by 3D rapid prototyping (Dimension 1200es, Breakaway Support Technology, USA). Polydimethylsiloxane (PDMS) (Sylgard 184, Dow Corning Corp., USA) prepared with base-to-crosslinker ratio of 9:1 was cast into the mold and cured at 80° C. for 30 minutes (FIG. 8A). Afterwards, the PDMS device was covalently bonded to glass coverslip with a thickness of 100 μm (GOLD SEAL cover glass, Electron Microscopy Sciences, USA) following standard O₂ plasma treatment process. The device was subsequently dip-coated in a 1% w/w Teflon AF 1600 (DuPont Corp., USA) in FC-40 solvent (3M Company, USA) and baked overnight at 80° C. Teflon coating was applied in order to protect against surface adsorption of biomolecules and to prevent spreading of reagent droplets on the cartridge surface. An alternative fabrication method utilized laser cutter to directly machine cartridges out of polymethylmethacrylate (PMMA), followed by laminating to an adhesive film and bonding to glass cover slip. The device was dip-coated in Teflon and baked overnight at 80° C. as mentioned. PMMA was selected for its biocompatibility, low cost and capacity for mass production. The completed prototype is shown in FIG. 1A.

### Cartridge Priming and Assay Protocol

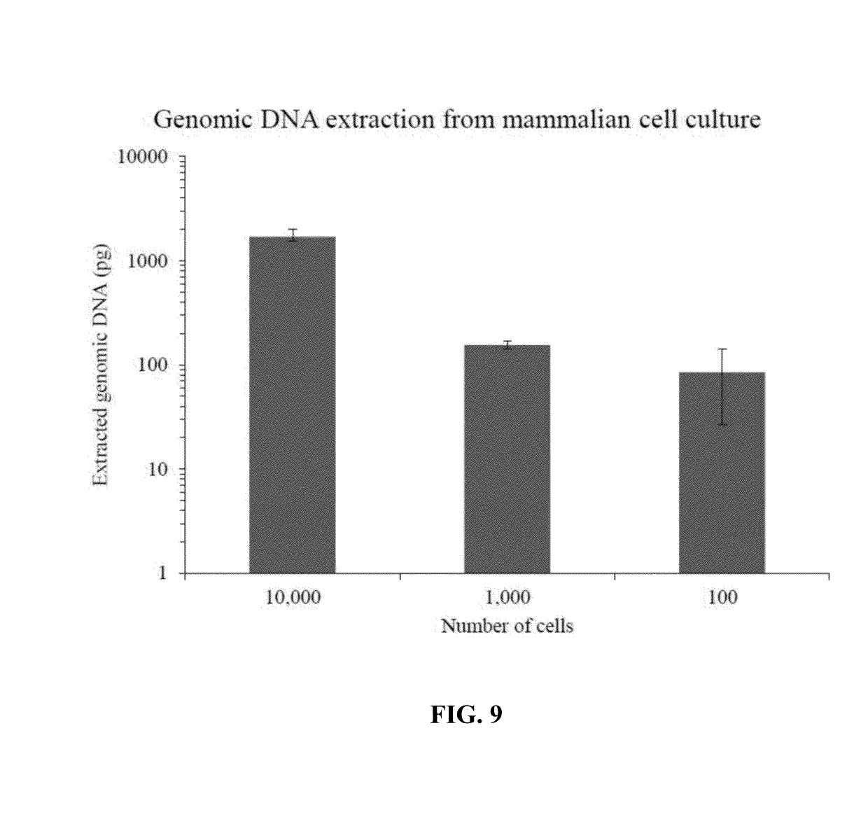

The on-chip procedure for the cell lysis, extraction and purification of nucleic acids followed standard protocol based on solid phase extraction methods using commercially available silica-coated magnetic particles. Reagents for nucleic acids extraction were purchased from Roche Diagnostic (MagnaPure LC DNA Isolation Kit, Roche Diagnostic Corporation, USA). Unspun human whole blood from male donor containing heparin anti-coagulant was purchased from Biological Specialty Corporation (Colmar, Pa., USA). The cartridge described previously was first filled with mineral oil (M5904, Sigma-Aldrich, USA) containing 0.5% w/w of surfactant Span-80 (Sigma-Aldrich, USA) to prevent the evaporation of reagents. Sessile reagent droplets (10-30 μL) corresponding to each step of the assay protocol were loaded sequentially into the compartments. 5 μL of whole blood was transferred into a mixture containing 10 μL lysis/binding buffer (LSB), 5 μL Tris-EDTA buffer, 1 μL Proteinase K (20 mM) and 3 μL of magnetic particles (Roche Isolation Kit), and the mixture was dispensed into the first compartment of the device. Compartments 2 through 6 were sequentially loaded with the following reagents: 15 μL washing buffer 1 (WB1a), 15 μL washing buffer 1 (WB1b), 15 μL washing buffer 2 (WB2a), 15 μL washing buffer 2 (WB2b), and 10 μL PCR reagent mixture, as illustrated in FIG. 1D. The lysis mixture in the first compartment was incubated on chip for 5 minutes with 100 cycles of agitation. Each washing step consisted of merging particle with the wash buffer, 15 cycles of agitation for 45 seconds, and splitting of particle into the next reagent droplet. Washing steps were followed by elution of genomic DNA into PCR mixture droplet by incubating the magnetic particles for 5 minutes with continuous agitation. The last compartment was intentionally left empty as a waste collection reservoir. PCR reagent mixture was prepared using QIAGEN HotStarTaq Core Kit (QIAGEN N.V., Netherlands). Total time required for sample preparation before PCR was 15 minutes, only a fraction of time required for manual extraction by a technician.³⁵ Using this protocol, the platform was found to be capable of retrieving approximately 100 pg of genomic DNA from as few as 100 cells (see FIG. 9).

### Magnetic Bead Manipulation System

FIG. 1B depicts the schematic assembly of the magnetic bead manipulation system. The fabricated device was attached on top of PCB containing a linear array of planar coils that were used as electromagnets to manipulate the magnetic beads. The sieve structures were aligned to the coils as shown in FIG. 1C to ensure proper droplet manipulation. The magnitude of coil-induced magnetic fields was controlled by modulating the driving current source, while the directions of fields generated by each coil were controlled by an H-bridge circuit. Custom software written in LabVIEW (National Instruments, USA) was used to sequentially control the magnitude, duration and direction of magnetic fields. Automated magnetic bead manipulation operations were achieved by programming a routine combining particle splitting and agitation operations in each reagent.

A set of permanent magnets were placed on top of a soft magnetic steel plate, generating a 50×10 mm plane of uniform transverse magnetic field measuring approximately 50 mT. Magnetic field gradients were adjusted by a two-layer, 200 μm thick PCB. The PCB contained 7 coils on the top layer and 6 coils on the bottom layers, in which there was a partial overlap of adjacent coils with the center-to-center distance of 3 mm. Each coil was designed in a square profile with 8 windings. The height, width, and pitch of the copper lines were 35 μm, 150 μm, and 150 μm, respectively.

A thermoelectric module (Custom Thermoelectric Inc, USA) was placed underneath the PCB as a cooling pad to alleviate the effects of Joule heating on thermally sensitive reagents such as the PCR mixture, which contain heat-activated enzymes. Cooling temperature was feedback controlled by using a commercial PID controller (Accuthermo Technology, USA) and a K-type thermocouple (Omega Engineering Inc, USA) mounted on the surface of the module.

### Nucleic Acid Amplification and Detection

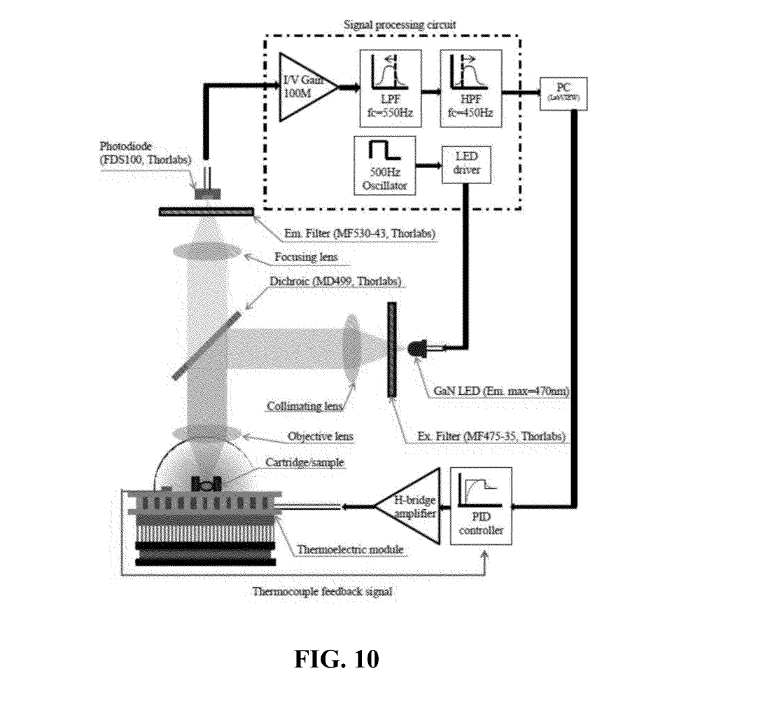

After sample preparation was performed using the magnetic droplet manipulation system, the cartridge was transferred to a custom-built instrument for thermal cycling and fluorescence detection (FIG. 10). Thermal cycling was performed using a thermoelectric module and PID controller in a similar arrangement used for Joule heat management. In order to account for transition times and temperature offsets between the surface of thermoelectric module and the droplet, thermal cycling conditions were calibrated to temperature profile obtained by monitoring a 10 μL PCR master mixture with a secondary thermocouple. Cycling parameters were as follows: thermal activation (86° C. for 15 minutes), 50 cycles of denaturing (86° C. for 80 seconds), annealing (60° C. for 100 seconds), elongation (72° C. for 80 seconds), followed by final elongation (72° C. for 3 minutes). Primers targeting codon 12 of exon 2 of the KRAS oncogene was used as previously described.³⁶ Primers were synthesized by Integrated DNA Technologies Inc. (USA) and 1×LC Green Plus+(Idaho Technologies Inc, USA) was used as the double-stranded DNA binding reporter dye. Primer sequences are presented in Table 1.

TABLE 1
Primer sequences used for real-time
amplification.

| Oligonucleotide | |
|---|---|
| name | Sequence |
| KRAS forward primer | 5′- TAAGGCCTGCTGAAAATGACTG -3′ |
| KRAS reverse primer | 5′- TGGTCCTGCACCAGTAATATGC -3′ |

Table 2 shows qualitative comparison of the droplet splitting capabilities in different buffers using varying amount of magnetic particles. Magnetic particle manipulation was visually inspected and categorized under five regimes: a) splitting operation is performed consistently without apparent issues; b) splitting is accompanied by either loss of magnetic particles in parent droplet or excessive carryover of parent reagent by daughter droplet; c) splitting is occasionally achieved; d) splitting failure characterized by poor dissociation of magnetic particles from parent droplet; e) splitting failure characterized by complete inability of magnetic particle to dissociate from parent droplet.

TABLE 2

| Magnetic | Lysis/binding | Washing | Washing | Washing | Washing | Elution |
|---|---|---|---|---|---|---|
| particle loading | buffer | buffer 1a | buffer 1b | buffer 2a | buffer 2b | buffer |
| 200 μg | V | V | Δ | X | n/a | n/a |
| 300 μg | V | V | V | V | V | V |
| 400 μg | ◯ | ◯ | V | V | V | V |
| 500 μg | ◯ | ◯ | ◯ | ◯ | ◯ | ◯ |
| 600 μg | ◯ | XX | n/a | n/a | n/a | n/a |

V: excellent splitting,
◯: good splitting but bringing much buffer or losing much beads,
Δ: unstable splitting,
X: splitting failed,
XX: breakup failed

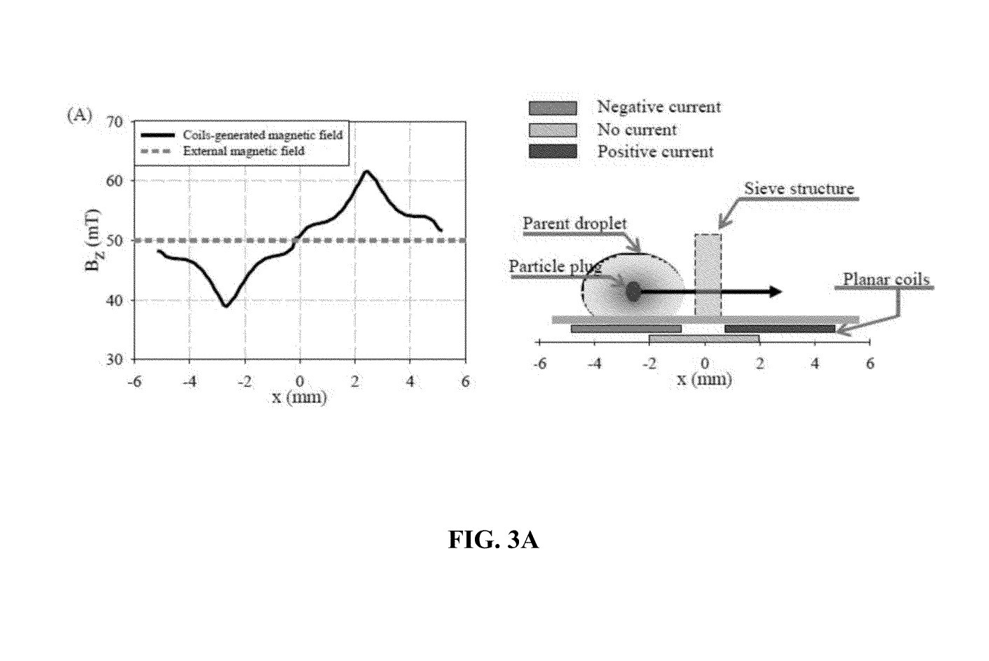

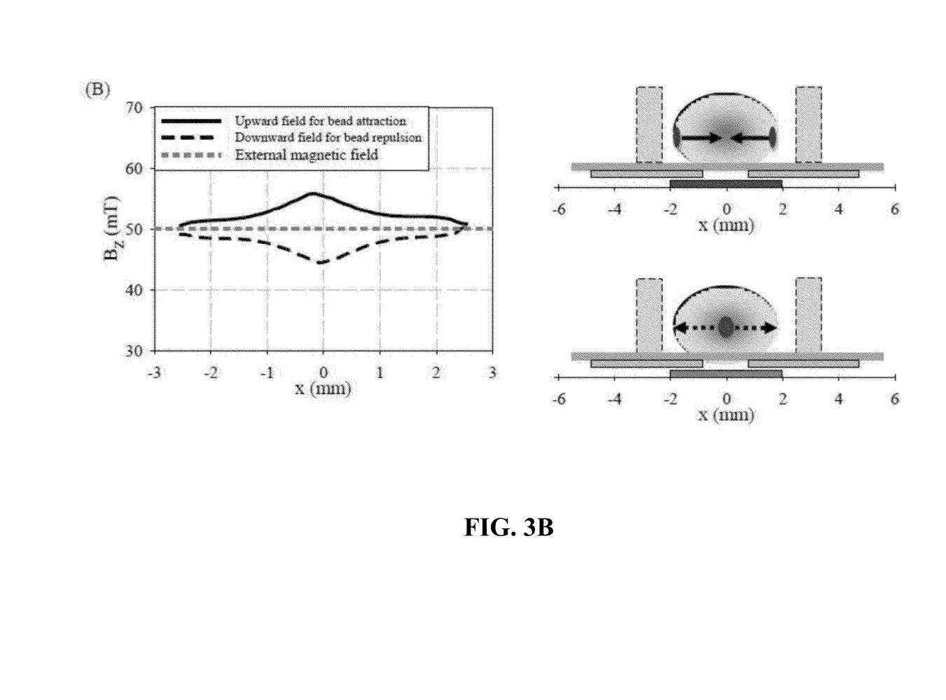

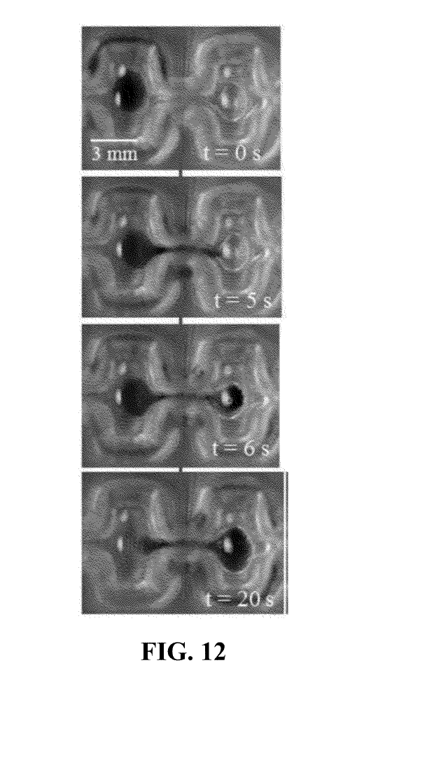

In the case of 300 μg particle load, assuming that all the particles were packed closely in the face-centered cubic configuration, the resulting capillary force was estimated to be in the range of several μN. When 3 A current is applied to the coils in the splitting configuration in FIG. 3A, field gradient of 4 T/m is generated. The compacted magnetic clusters result in a larger pressure, which can impose sufficient magnetic force on the droplet wall for breaking up the droplet. When the load is below 300 μg, the splitting performance is reduced. Droplet splitting may fail in washing buffer 2 because the magnetic force induced by the less magnetic beads cannot overcome the relatively higher capillary force due to this reagent having a higher interfacial tension. Meanwhile, in the case of 600 μg load, the splitting failed because the entire parent droplet was brought to the next droplet by the large magnetic bead clusters, as shown in FIG. 12. In conclusion, particle load should be selected in the range between 300-500 μg for droplet manipulation.

Fluorescence was detected using a custom-built, portable optical instrument in epifluorescence configuration consisting of a blue light-emitting diode (LED) source (λₘₐₓ=470 nm) and photodiode. Briefly, pulsed light from LED was passed through an excitation filter, reflected by a dichroic mirror, and was then focused onto the PCR reagent mixture droplet. Fluorescence emission from the reporter dye bound to genomic target was passed through an emission filter and focused onto a frequency-sensitive detector. The signals from the detector were recorded using analog-to-digital acquisition (USB-6229, National Instruments, USA) at a 5-second interval with a bin time of 150 ms. Signals obtained over the last 25 seconds of each annealing phase were averaged and plotted to generate a real-time amplification profile.

Agarose gel electrophoresis was performed to verify product amplified on droplet platform. Gels were run at 8 V/cm for 60 minutes. DNA was stained using GelStar nucleic acid gel stain (Lonza Rockland Inc, USA) and scanned under an epi-illumination configuration using a Kodak Gel Logic 200 Imaging System (Kodak, USA).

### Results and Discussion

Operating Procedure

In this droplet cartridge (FIG. 1D), genomic DNA extraction from whole blood and subsequent real-time amplification were integrated into a single process on the cartridge. First, whole blood sample was loaded to the initial compartment containing lysis buffer mixture and incubated. During this process, the white blood cells in sample matrix were lysed to release the genomic material. The released genomic DNA was adsorbed to silica-coated surface of magnetic particles in the presence of chaotropic salt in the lysis/binding buffer. After incubation, magnetic particles were split from the lysis buffer using coil-induced field gradient and merged into a series of washing buffers. The magnetic particles were then merged with the last droplet reagent containing PCR mixture and incubated. The lower concentration of salt present in a PCR mixture enables the genomic DNA bound on the particle surface to be released into the mixture. Afterwards, the magnetic particles were split from the PCR mixture into the waste chamber and the entire cartridge was loaded onto a custom-built thermal cycler and optical detection instrument for real-time amplification on chip.

### Droplet Manipulation Platform

Droplet kinematic behaviors were governed by the interaction between two forces. The first was the magnetic force acting on the magnetic particles within the droplet, while the second was the capillary force induced by droplet deformation.³⁴ The proposed droplet manipulation strategy employed on-cartridge topographic features to control capillary forces, with an external actuation mechanism to control magnetic force on particles. Actuation of magnetic particles is realized on this platform using planar coil-induced magnetic field gradients in presence of uniform static field.²³ Briefly, this mechanism utilizes a large, uniform transverse magnetic field B₀ to strongly polarize the magnetic moments of magnetic particles (FIG. 2A). Planar coils are used to induce small magnetic fields in parallel with B₀. Since the magnetic force acting on each particle is described by the dot product of magnetic moment and the applied field gradient,³⁷ force is generated towards the direction where the transverse component (B_{z}) of field generated by planar coils is maximized. Controlling the direction of driving current allows switching of the polarity of coil-induced field, resulting in magnetic field gradient that can be made both positive and negative. This enables both attractive and repulsive forces which is used to perform transport, splitting and agitation of magnetic particles.

Meanwhile, capillary force is controlled using sieve structures to facilitate droplet splitting. Capillary force has two components, the Laplace force and the interfacial tension force.³⁸ As illustrated in FIG. 2A, sieve structures were incorporated between reagent compartments to facilitate droplet deformation during splitting. When the magnetic droplet was actuated through the sieve, the droplet was deformed to attain negative mean curvature resulting in negative Laplace force. This phenomenon mitigates the effective capillary force, enabling droplet fission with a relatively low magnetic force.

### Droplet Splitting and Merging

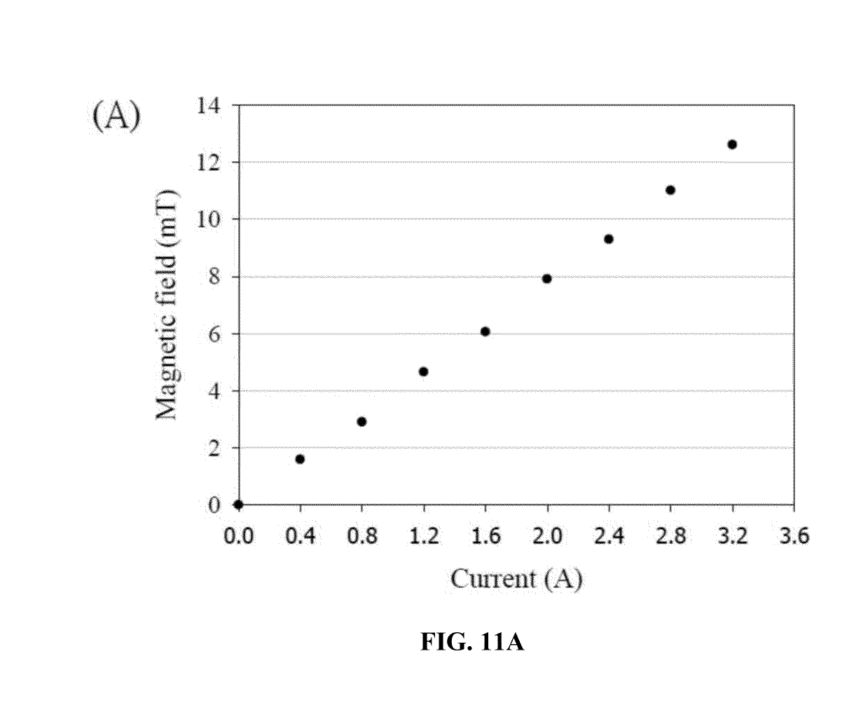

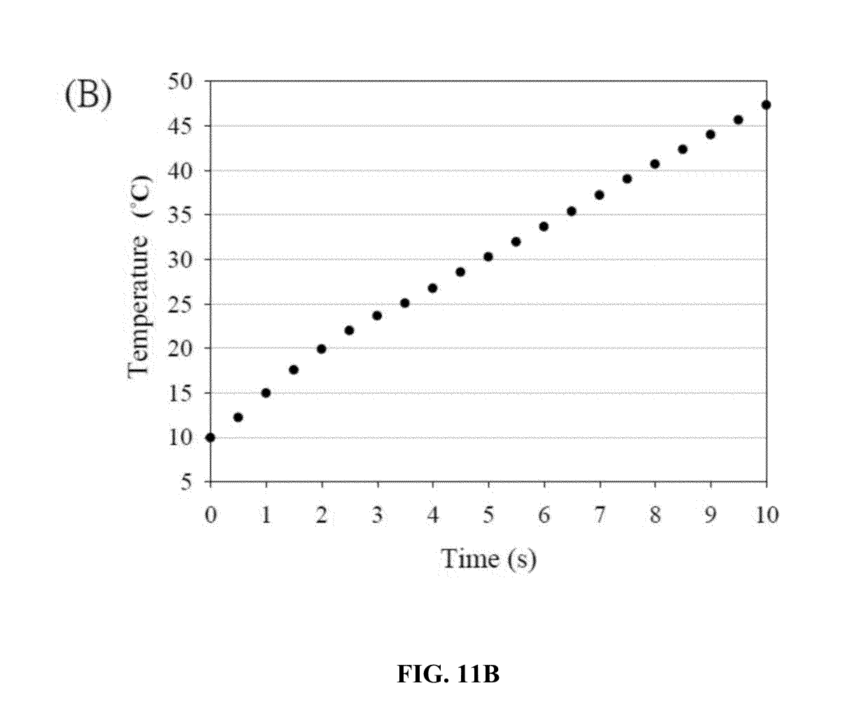

To split magnetic particles from the droplet with maximal retention of particles, a magnetic field gradient was generated by applying a positive, zero, and negative current to three consecutive coils, respectively (see FIG. 3A). The magnetic fields were simulated numerically using commercial software (CFD-ACE+, CFD-RC, USA). FIG. 2B shows the designed field gradient was estimated to be 4 T/m with an applied current of 3 A, which was consistent with experimentally obtained values (FIG. 11A). The resulting magnetic force was sufficient to overcome the capillary force and cause droplet fission. In order to mitigate the effects of Joule heating on interfacial tensions and thermally sensitive reagents such as the PCR mixture, the temperatures inside the droplet during the splitting process were maintained below 30° C. by using a thermoelectric cooler located below the PCB (FIG. 11B).

Droplet splitting is primarily influenced by the interfacial tension between the droplet and the surrounding medium, as well as the magnetic force. In the current assay there were three levels of interfacial tensions between different reaction droplets and surrounding oil medium: 1) lysis mixture containing the lysis/binding buffer, which includes 20-30% w/w Triton X-100, 2) washing buffers 1 and 2 containing 30-60% w/w ethanol, and 3) the PCR master mixture mostly composed of water and salts. Experimental conditions were optimized such that consistent splitting and merging could be achieved in all three types of reagents. Splitting conditions were adjusted by varying three factors: 1) magnetic particle load, 2) surfactant concentration in surrounding oil medium, and 3) sieve gap. Topography-assisted splitting enabled us to apply a wide range of particle load to optimize the splitting conditions simultaneously for the reagents with several different interfacial tensions. If particle load was less than 200 μg, the magnetic force applied to the beads was insufficient to overcome interfacial tension of the parent droplet. With particle load in excess of 600 μg, the separated bead plug carried a substantial amount of buffer from the parent droplet. Particle loads between these limits were examined to qualitatively assess the splitting capability from various reagents (see Table 1). Higher surfactant concentration was observed to cause droplet instability indicated by difficulty in priming the device with reagents, while lower surfactant concentration was accompanied by increased difficulty in splitting. Details of the optimization process are included in Table 1.

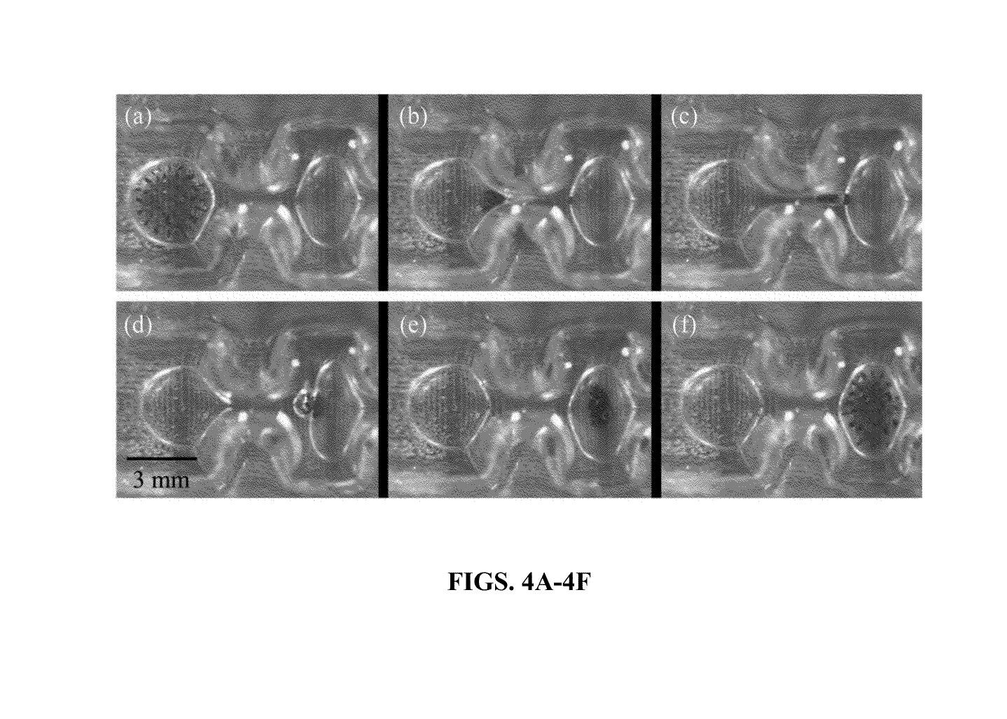

FIG. 4 shows a photographic sequence of surface topography-assisted droplet splitting process, where a droplet containing magnetic particles is deformed through the sieve structures and dissociated into a small plug carrying trace amount of aqueous material from the parent droplet. As field maximum was generated at the destination compartment, magnetic particles were collected into a plug and pulled through the sieve structure until scission occurred at the elongated neck (see FIG. 4D). After splitting from the parent droplet, magnetic particles were merged with the subsequent droplet. At the optimal concentration of surfactants, magnetic particles could be split from the parent droplet and transported to the next compartment without prematurely merging with the subsequent droplet. The resulting conditions enabled the droplet compartments to be packed more compactly on a smaller cartridge footprint. Droplet merging could be actively induced by generating field maximum at the center of current compartment, resulting in collection of magnetic particles at the center of the droplet (FIG. 4E).

### Particle Agitation

One of the advantages of this platform is that efficient mixing can be achieved by agitating magnetic particles under alternating attraction and repulsion forces, as illustrated in FIG. 3B. Particle agitation involves a single coil centered on the droplet. When the direction of coil-generated magnetic field is parallel to background field, the maximal field occurs at the center of coil and particles are concentrated at the center of droplet. When the current is reversed, coil-generated field is antiparallel to background field; the center of droplet becomes a local field minimum, and particles are dispersed towards the fringes of the droplet. The particles can be repeatedly agitated inside the droplet by alternating the polarity of current.

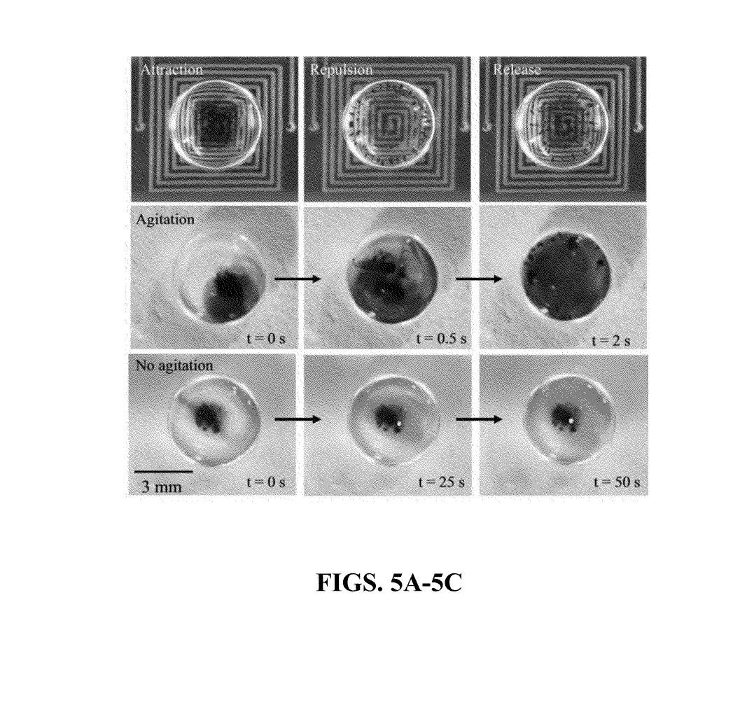

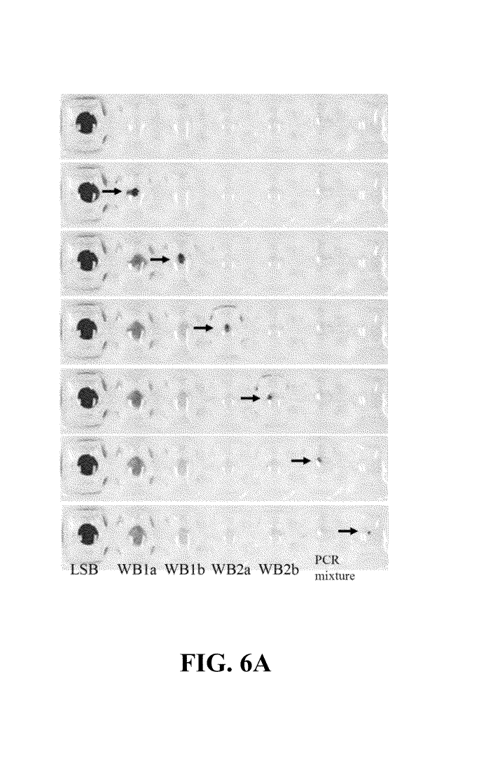

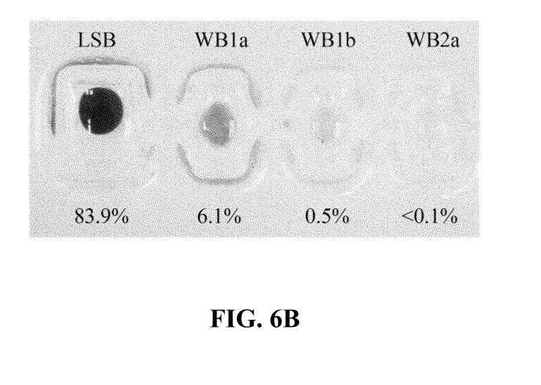

In this work, particle agitation was performed by applying a current of 1.5 A with alternating frequency of 0.5 Hz. Performance was evaluated by comparing the diffusion process of the dark blue food dye in water in presence and absence of agitation Inner convective flow was introduced with the assistance of agitation and accelerated the mixing process, as shown in FIG. 5B. Mixing of food dye with water was achieved in 2 seconds. Conversely, hardly any mixing was observed over the span of 50 seconds when the process was left to diffusion alone, as shown in FIG. 5C. In contrast to the permanent magnet-based actuation scheme, in which the magnetic particles are anchored to the surface due to magnetic attraction or higher specific gravity, mixing efficiency can be enhanced by means of magnetic agitation in the proposed system.

### Particle Washing

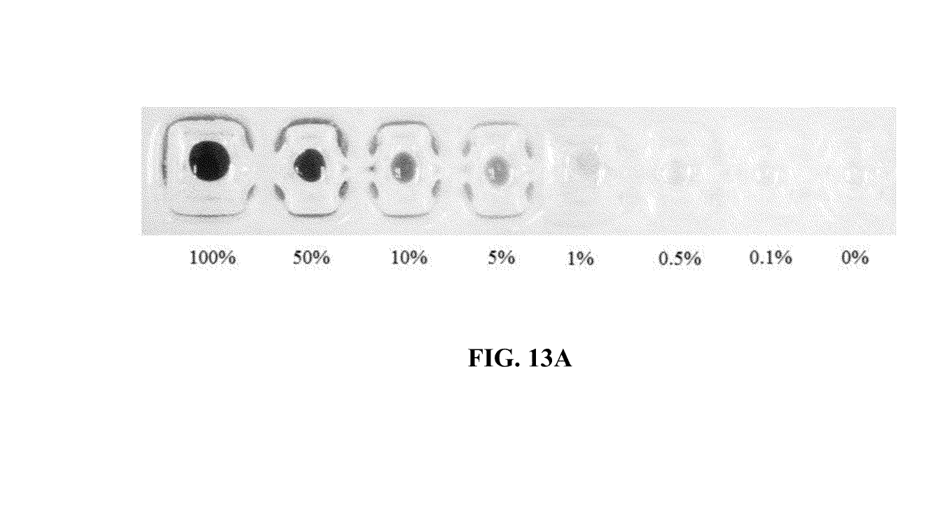

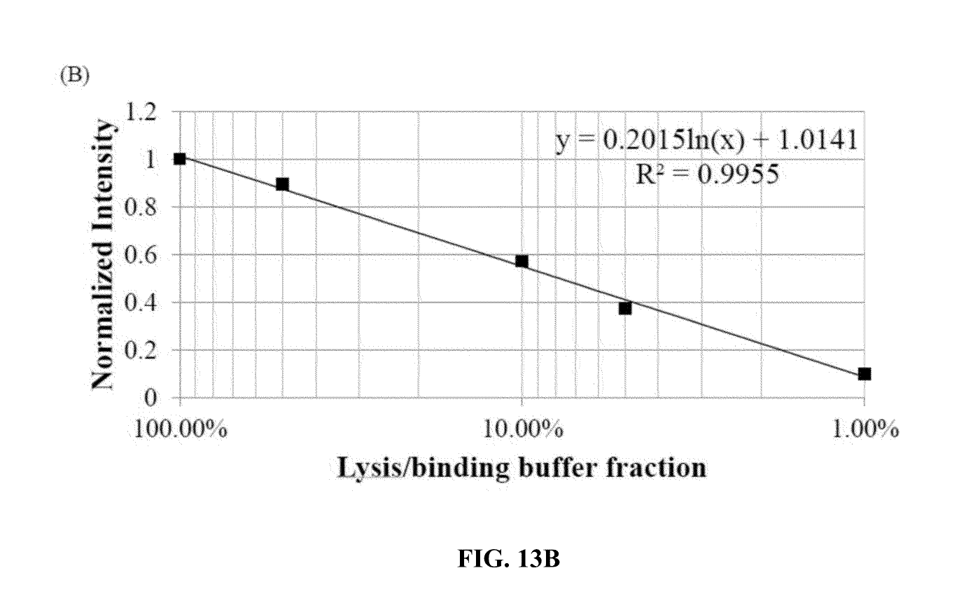

When crude biological samples are lysed, the buffer carried over by magnetic clusters contain PCR inhibitors including hemo-globin and DNA-binding proteins.³⁹ Performance of washing process was evaluated by estimating the volume of buffers being carried over between each reagent droplet. Washing process was defined as a combination of droplet splitting with subsequent merging. Owing to the presence of a dark blue dye in lysis/binding buffer, it is a suitable indicator of cleanness after each washing step. A reference curve was first generated by measuring the mean gray value of color of lysis/binding buffer against serial dilutions to evaluate the effect of washing (see FIG. 13B). Afterwards, the magnetic particles were split from LSB droplet and were subsequently washed in WB1a, WB1b, WB2a, and WB2b, as demonstrated in FIG. 13A. FIG. 13B presents the mapped grey values of LSB, WB1a, WB1b, and WB2a on the reference curve with dye concentrations of 83.9% (CLSB), 6.1% (CWB1a), 0.5% (CWB1b), and less than 0.1% (CWB2a), respectively.

Following a simple dilution calculation (VC×CLSB=CWB1a×(VC+V_{droplet})),³² the carryover volume (VC) after the first wash step was estimated to be around 1.2 μL that is merged with a subsequent droplet of a volume (V_{droplet}) of 15 μL. Similarly, the carryover volume after the second washing was estimated to be around 1.3 μL. Volume occupied by the magnetic particles (<0.1 μL) was considered to be negligible. It is reasonable to estimate that each washing on the device step results in >10 fold dilution, meaning that the lysis buffer residue and PCR inhibitors carried alongside magnetic particles are attenuated 10⁴-fold or greater in the PCR reaction buffer after the four washing steps.

### On-Chip Real-Time PCR

As demonstrated, the platform is capable of automated processing of whole blood sample into a qPCR-ready droplet on a single cartridge. Real-time amplification detection of the KRAS oncogene was performed on the droplet platform using 5 μL of human whole blood as the biological sample input. Genomic DNA was first isolated from whole blood using automated processing described in an earlier section and eluted directly into a PCR reagent mixture droplet containing 1×LCGreen+. The processed cartridge was subsequently mounted on a custom-built, portable thermal cycler module with an approximate heating rate of 1.2° C.s⁻¹ and cooling rate of 2.8° C.s⁻¹. Hold times in cycling parameters included transition times between temperature zones.

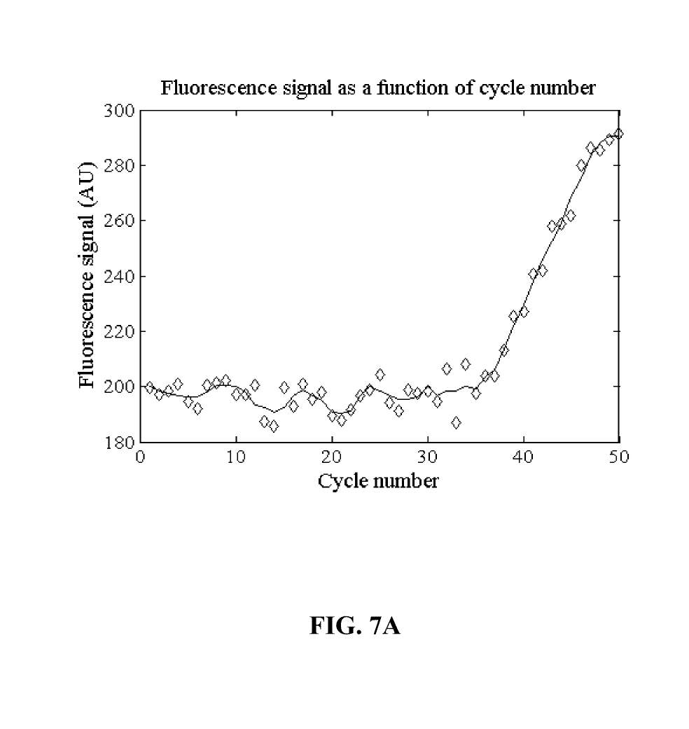

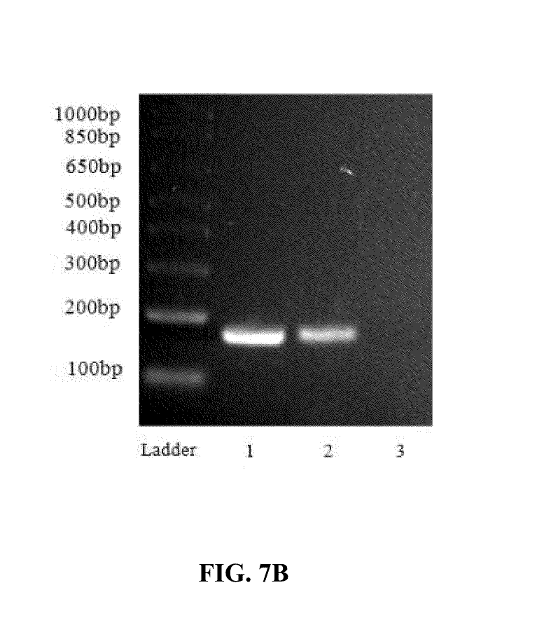

Denaturation was set at 86° C. in order to alleviate issues regarding evaporation of various reagents on the cartridge. Denaturation of double-stranded DNA at a temperature range of 93˜95° C. as typically performed in conventional PCR is primarily for complete denaturation of longer genomic DNA fragments in earlier cycles, and amplification can take place normally for shorter products using lower denaturation temperatures.⁴⁰ Since lower denaturation temperature also reduces enzyme inactivation, product yield is also enhanced and amplification can be performed over an extended number of cycles.⁴⁰ FIG. 7A shows the real-time PCR signal from KRAS detected within the droplet. We observed a high cycle number that was attributed to lower amplification efficiency due to long transition times between temperature zones. Thermal cycling efficiency can be improved by further optimizing temperature controller parameters and cartridge design in the future.

In order to verify that the amplification signals were associated with the target region rather than primer dimers or unspecific products, the amplified product was analyzed using 2% agarose gel electrophoresis. Positive control samples were generated by thermal cycling PCR mixtures spiked with 2 ng male genomic DNA (Promega Corporation, USA) under the same cycling conditions on a conventional thermal cycler, while negative control was generated by thermal cycling the same mixture without genomic targets (no template control). As shown in FIG. 7B, gel results show that the products were of expected length (Lane 1), which confirmed amplification of target product from the sample.

### CONCLUSIONS

The above demonstrates an example of an automated magnetic droplet-based system according to an embodiment of the current invention for whole blood genetic testing, integrating nucleic acid extraction from crude samples, nucleic acid amplification, and real-time fluorescence detection on a single disposable cartridge. Topographical barriers on the cartridge were used in tandem with magnetic coil-based instrument to create a simple and efficient droplet manipulation scheme. Planar coil structures provided a ubiquitous actuation mechanism for the splitting, transport and agitation/mixing of magnetic particles, while topographical barriers enabled efficient splitting and isolation of reagent droplets with varying interfacial tensions. Using this platform, automated genomic DNA extraction from whole blood was achieved in 15 minutes. The cartridge was integrated with external thermal cycling and optical detection modules to successfully demonstrate real-time amplification detection of genetic target. A major strength of the droplet manipulation platform can be its flexibility, as the platform can simultaneously handle a diverse range of reagents and is also amenable to integration with thermal control and optical detection. Some embodiments of the current invention could deliver nucleic-acid based diagnostic assays for a point-of-care setting, for example.

Some aspects of the current invention can be directed to the following:
Method to fabricate microfluidic cartridge for storage of reagents, extraction of nucleic acids from crude samples, and PCR for DNA detection.
Method to fabricate microfluidic cartridge incorporating topographical barriers to generate regions with different surface tensions.
Method to split the SSP from the droplet with the assistance of the topographical barrier which creates larger surface tension to hold the droplets, while the SSP can pass through the gap between barriers
Method to actuate SSP using planar coil structures embedded in a printed circuit board (PCB) to facilitate splitting, merging, transport and mixing in various reagents.
A self-sustained cartridge where buffers and reagents required are stored in the form of droplet inside a chamber filled with mineral oil, eliminating the need for tubing connected to external reagents.
Pre-stored buffer droplets are restrained by the surface topological features, which hold the droplet in position and prevent the droplet from moving and merging with each other.
Filling the chamber with the oil provides thermal isolation to prevent the evaporation of the droplets. It also provides physical isolation of the potential biohazardous sample from the environment.
Device with prepackaged reagent is sealed with adhesive tape. By simply peeling off the tape, the device is ready to use, which greatly simplifies the operation for point-of-care applications.
PCR detection of genetic biomarkers is performed directly from crude sample input on the same platform and all procedures are performed in the form of droplets with the help of a PCB electromagnet array.
An integrated miniaturized fluorescence detection system that is insensitive to the ambient optical noise.

### REFERENCES

(1) Mark, D.; Haeberle, S.; Roth, G.; von Stetten, F.; Zengerle, R. Chem Soc Rev. 2010. 39(3): 1153-1182.
(2) Zhang, Y.; Park, S.; Yang, S.; Wang, T. H. Biomed Microdevices. 2010. 12(6): 1043-1049.
(3) Sauer-Budge, A. F.; Mirer, P.; Chatterjee, A.; Klapperich, C. M.; Chargin, D.; Sharon, A. Lab Chip 2009. 9(19): 2803-2810.
(4) Burns, M. A.; Johnson, B. N.; Brahmasandra, S. N.; Handique, K.; Webster, J. R.; Krishnan, M.; Sammarco, T. S.; Man, P. M.; Jones, D.; Heldsinger, D.; Mastrangelo, C. H.; Burke, D. T. Science 1998. 282(5388): 484-487.
(5) Pal, R.; Yang, M.; Lin, R.; Johnson, B. N.; Srivastava, N.; Razzacki, S. Z.; Chomistek, K. J.; Heldsinger, D. C.; Haque, R. M.; Ugaz, V. M.; Thwar, P. K.; Chen, Z.; Alfano, K.; Yim, M. B.; Krishnan, M.; Fuller, A. O.; Larson, R. G.; Burke, D. T.; Burns, M. A. Lab Chip 2005. 5(10): 1024-32.
(6) Easley, C. J.; Karlinsey, J. M.; Bienvenue, J. M.; Legendre, L. A.; Roper, M. G.; Feldman, S. H.; Hughes, M. A.; Hewlett, E. L.; Merkel, T. J.; Ferrance, J. P.; Landers, J. P. Proc Natl Acad Sci USA 2006. 103(51): 19272-7.
(7) Lien, K. Y.; Lee, W. C.; Lee, H. Y.; Lee, G. B. Biosens Bioelectron., 2007. 22(8): 1739-48.
(8) Mariella, Jr., R. Biomed Microdevices 2008. 10(6): 777-84.
(9) Park, S.; Zhang, Y.; Lin, S.; Wang, T. H.; Yang, S. Biotechnol Adv. 2011. 29(6): 830-9.
(10) Abdelgawad, M.; Wheeler, A. R. Adv. Mater. 2009. 21(8): 920-925
(11) Pollack, M. G.; Pamula, V. K.; Srinivasan, V.; Eckhardt, A. E. Expert Rev Mol Diagn. 2011. 11(4): 393-407.
(12) Issadore, D.; Franke, T.; Brown, K. A.; Westervelt, R. M. Lab Chip 2010. 10(21): 2937-43.
(13) Fan, S. K.; Hsieh, T. H.; Lin, D. Y. Lab Chip 2009. 9(9): 1236-42.
(14) Velev, O. D.; Prevo, B. G.; Bhatt, K. H. Nature 2003. 426(6966): 515-6.
(15) Hunt, T. P.; Issadore, D.; Westervelt, R. M. Lab Chip 2008. 8(1): 81-7.
(16) Boles, D. J.; Benton, J. L.; Siew, G. J.; Levy, M. H.; Thwar, P. K.; Sandahl, M. A.; Rouse, J. L.; Perkins, L. C.; Sudarsan, A. P.; Jalili, R.; Pamula, V. K.; Srinivasan, V.; Fair, R. B.; Griffin, P. G.; Eckhardt, A. E.; Pollack, M. G. Anal Chem.
2011. 83(22): 8439-47.
(17) Welch, E. R.; Lin, Y. Y.; Madison, A.; Fair, R. B. Biotechnol J. 2011. 6(2): 165-76.
(18) Srinivasan, V.; Pamula, V. K.; Fair, R. B. Lab Chip 2004. 4(4): 310-5.
(19) Schaller, V.; Sanz-Velasco, A.; Kalabukhov, A.; Schneiderman, J. F.; Oisjoen, F.; Jesorka, A.; Astalan, A. P.; Krozer, A.; Rusu, C.; Enoksson, P.; Winkler, D. Lab Chip 2009. 9(23): 3433-6.
(20) Sista, R.; Hua, Z.; Sudarsan, A.; Srinivasan, V.; Eckhardt, A.; Pollack, M. G.; Pamula, V. K. Lab Chip 2008. 8(12): 2091-104.
(21) Hua, Z.; Rouse, J. L.; Eckhardt, A. E.; Srinivasan, V.; Pamula, V. K.; Schell, W. A.; Benton, J. L.; Mitchell, T. G.; Pollack, M. G. Anal Chem. 2010. 82(6): 2310-6.
(22) Kim, H.; Bartsch, M. S.; Renzi, R. F.; He, J.; Van de Vreugde, J. L.; Claudnic, M. R.; Patel, K. D. J Lab Autom. 2011. 16(6): 405-14.
(23) Rida, A.; Fernandez, V.; Gijs, M. A. M. Appl. Phys. Lett. 2003. 83(12): 2396.
(24) Okochi, M.; Tsuchiya, H.; Kumazawa, F.; Shikida, M.; Honda, H. J Biosci Bioeng. 2010. 109(2): 193-197.
(25) Ohashi, T.; Kuyama, H.; Hanafusa, N.; Togawa, Y. Biomed Microdevices 2007. 9(5): 695-702.
(26) Pipper, J.; Inoue, M.; Ng, L. F-P.; Neuzil, P.; Zhang, Y.; Novak, L. Nat. Med. 2007. 13(10): 1259-1263.
(27) Shikida, M.; Takayanagi, K.; Inouchi, K.; Honda, H.; Sato, K. Sens. Actuators B Chem. 2006. 113(1): 563-569.
(28) Tsuchiya, H.; Okochi, M; Nagao, N.; Shikida, M.; Honda, H. Sens. Actuators B Chem. 2008. 130(2): 583-588.
(29) Zhang, Y.; Park, S.; Liu, K.; Tsuan, J.; Yang, S.; Wang, T. H.; Lab Chip 2011. 11(3): 398-406.
(30) Pan, X.; Zeng, S.; Zhang, Q.; Lin, B.; Qin, J. Electrophoresis 2011. 32(23): 3399-3405.
(31) Beyzavi, A.; Nguyen, N. T. J. Phys. D: Appl. Phys. 2009. 42(1): 015004.
(32) Lehmann, U.; Vandevyver, C.; Parashar, V. K.; Gijs, M. A. M. Angew. Chem. Int. Ed. 2006. 45(19): 3062-3067.
(33) Lehmann, U.; de Courten, D.; Vandevyver, C.; Parashar, V. K.; Gijs, M. A. M. Microelectron. Eng. 2007. 84(5-8): 1669-1672.
(34) Long, Z. C.; Shetty, A. M.; Solomon, M. J.; Larson, R. G. Lab Chip 2009. 9(11): 1567-1575.
(35) Dundas, N.; Leos, N. K.; Mitui, M.; Revell, P.; Rogers, B. B. J. Mol. Diagn. 2008. 10(4): 311-31637.
(36) Ho, C. L.; Kurman, R. J.; Dehari, R.; Wang, T.; Shih, I. M. Cancer Res. 2004. 64(19): 6915-6918.
(37) Shevkoplyas, S. S.; Siegel, A. C.; Westervelt, R. M.; Prentiss, M. G.; Whitesides, G. M. Lab Chip 2007. 7(10): 1294-1302.
(38) Chau, A.; Rignier, S.; Delchambre, A.; Lambert, P. Modelling Simul. Mater. Sci. Eng. 2007. 15(3): 305-317.
(39) Wilson, I. G. Appl. Environ. Micobiol. 1997. 63(10): 3741-3751.
(40) Yap, E. P.; McGee, J. O. Nucleic Acids Res. 1991. 19(7): 1713.
   The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A microfluidic system, comprising:
a microfluidic cartridge; and
an electromagnetic droplet actuator arranged proximate said microfluidic cartridge,
wherein said microfluidic cartridge comprises a plurality of droplet wells with topological barrier structures between adjacent wells,
wherein said topological barrier structures are configured to allow magnetic particles and material attached to said magnetic particles to pass between adjacent wells while confining droplets within respective wells, and
wherein said electromagnetic droplet actuator comprises a plurality of electromagnetic components arranged to provide an electronically selectable magnetic field pattern to actuate movement of a plurality of magnetic particles when contained within at least one droplet in at least one of said plurality of droplet wells.

2. A microfluidic system according to claim 1, wherein said electromagnetic droplet actuator is configured to provide a mixing mode such that said plurality of magnetic particles are caused to move with a time-varying pattern within said droplet to cause mixing within said droplet.

3. A microfluidic system according to claim 1, wherein said electromagnetic droplet actuator is configured to provide a separation mode such that said plurality of magnetic particles are caused to move from one droplet well to an adjacent droplet well along with material attached to at least some of said plurality of magnetic particles while each said droplet remains confined within a respective droplet well.

4. A microfluidic system according to claim 2, wherein said electromagnetic droplet actuator is configured to provide a separation mode such that said plurality of magnetic particles are caused to move from one droplet well to an adjacent droplet well along with material attached to at least some of said plurality of magnetic particles while each said droplet remains confined within a respective droplet well.

5. A microfluidic system according to claim 1, wherein said plurality of electromagnetic components are electromagnetic coils arranged such that each droplet well has a corresponding closest electromagnetic coil substantially centered thereon.

6. A microfluidic system according to claim 1, wherein said plurality of electromagnetic components comprise a first plurality of electromagnetic coils arranged such that each droplet well has a corresponding closest electromagnetic coil substantially centered thereon, and
wherein said plurality of electromagnetic components comprise a second plurality of electromagnetic coils arranged interstitially with respect to said first plurality of electromagnetic coils.

7. A microfluidic system according to claim 1, further comprising a magnet component arranged proximate said electromagnetic droplet actuator,
wherein said magnet component comprises at least one permanent magnet configured to provide a stationary magnetic field component to supplement said electronically selectable magnetic field pattern when produced by said electromagnetic droplet actuator.

8. A microfluidic system according to claim 1, further comprising a heat control unit arranged to be in thermal exchange with said microfluidic cartridge.

9. A microfluidic system according to claim 8, wherein said heat control unit comprises a thermoelectric cooler.

10. A microfluidic system according to claim 9, wherein said heat control unit further comprises a heat sink.

11. A microfluidic system according to claim 10, wherein said heat control unit further comprises a fan.

12. A microfluidic system according to claim 7, further comprising a heat control unit arranged to be in thermal contact with said microfluidic cartridge.

13. A microfluidic system according to claim 12, wherein said heat control unit comprises a thermoelectric cooler.

14. A microfluidic system according to claim 13, wherein said heat control unit further comprises a heat sink.

15. A microfluidic system according to claim 14, wherein said heat control unit further comprises a fan.

16. A method of processing a sample, comprising:
providing a droplet containing said sample and a plurality of magnetic particles in a droplet well of a microfluidic cartridge, wherein said microfluidic cartridge comprises a plurality of droplet wells with topological barrier structures between adjacent wells; and
applying a magnetic field pattern to actuate movement of said plurality of magnetic particles when contained within at least one droplet in at least one of said plurality of droplet wells,
wherein said topological barrier structures are configured to allow magnetic particles and material attached to said magnetic particles to pass between adjacent wells while confining droplets within respective wells.

17. A method of processing a sample according to claim 16, wherein said applying said magnetic field pattern to actuate movement of said plurality of magnetic particles causes said plurality of magnetic particles to move with a time-varying pattern within said droplet to cause mixing within said droplet.

18. A method of processing a sample according to claim 16, wherein said applying said magnetic field pattern to actuate movement of said plurality of magnetic particles causes said plurality of magnetic particles to move from one droplet well to an adjacent droplet well along with material attached to at least some of said plurality of magnetic particles.
